(19) United States
(12) Patent Application Publication
(10) Pub. No.: US 2008/0038208 A1
(43) Pub. Date: Feb. 14, 2008

(54) Pharmaceutical/cosmetic compositions comprising the lysine-d-proline-valine tripeptide

(75) Inventors: Yann Mahe, Morsang Sur Orge (FR); Bruno Buan, Bagnolet (FR)

Correspondence Address: BUCHANAN, INGERSOLL & ROONEY PC, POST OFFICE BOX 1404, ALEXANDRIA, VA, 22313-1404, US

(73) Assignee: SOCIETE L'OREAL S.A.

(21) Appl. No.: 11/907,565

(22) Filed: Oct. 15, 2007

Related U.S. Application Data
(63) Continuation of application No. 10/868,198, filed on Jun. 16, 2004.
Application No. 10/868,198 is a continuation of application No. 08/716,531, filed on Sep. 19, 1996, now abandoned.

(30) Foreign Application Priority Data
Sep. 19, 1995 (FR) 95-10977

(51) Int. Cl. A61K 38/06; A61K 8/64; A61K 9/06; A61K 9/107; A61K 9/12; A61P 29/00

(52) U.S. Cl. 424/45; 424/49; 424/59; 514/18

(57) ABSTRACT

Pharmaceutical/cosmetic compositions comprising a therapeutically effective amount of at least one peptide containing the lysine-proline-valine tripeptide sequence, the proline moiety of which existing in its dextrorotatory optical isomer form (DPro), or functional biological equivalent thereof, are well suited for the treatment of inflammation disease states afflicting a mammalian subject.

### CROSS REFERENCE TO RELATED APPLICATIONS

This application is a continuation of U.S. patent application Ser. No. 10/868,198, filed on Jun. 16, 2004, in turn a continuation of U.S. patent application Ser. No. 08/716,531, filed on Sep. 19, 1996, now abandoned, in turn, claiming priority to French Application No. 95-10977, filed on Sep. 19, 1995, disclosures of all of which are incorporated herein by reference in their entirety.

### BACKGROUND OF THE INVENTION

1. Technical Field of the Invention

The present invention relates to pharmaceutical/cosmetic compositions comprising a pharmaceutically effective amount of at least one peptide containing the lysine-proline-valine tripeptide, or any functional biological equivalent thereof, in a physiologically/pharmaceutically acceptable carrier therefor, in which the proline residue is in the form of its dextrorotatory optical isomer, for the treatment of inflammation.

2. Description of the Prior Art

Inflammation is a set of biological reactions which exists throughout the animal kingdom. In man, two patients out of three exhibit an inflammatory syndrome. The inflammation may be localized. It may be defined as the first response to any local attack by a series of non-specific reactions triggered whatever the initial cause and occurring in three steps: vascular, cellulo-vascular and tissue fibrosis. Swelling, pain, redness and warmth are the terms which may be used to describe localized inflammation. These are generally due to infiltration of the injured tissues by an oedema and/or to vasodilation of the capillaries.

The signs of inflammation can extend to fever, a state of general malaise and/or an increase in the concentration of certain blood plasma proteins. This is a phenomenon which entails, inter alia, a series of local cell reactions and the release of cytokines and other mediators such as substance P, prostaglandins, histamine or alternatively serotonin. It is manifested by a modification in the blood stream with, at the site afflicted, an increase in the vascular permeability, resulting in an escape of plasma proteins and of cells towards the extracelluar fluid and an extravasation of leukocytes, principally neutrophilic leukocytes, and macrophages towards the inflammatory site.

These phenomena are, in fact, the result of the action of the mediators of inflammation.

Among the factors involved in these inflammatory phenomena, exemplary are the cytokines, including in particular interleukin-1α, interleukin-1β, interleukin-6 or tumor necrosis factors α and β (TNF-α and -β), chemokines, such as interleukin-8 or monocyte chemotactic and activating factor (MCAF), or alternatively other chemotactic factors responsible for the recruitment of lymphocytic, monocytic, Langerhans or basophilic cells at the inflammatory site, such as leukotrienes B₄, or else other factors involved in the inflammatory cascade, such as arachidonic acid or prostaglandins, including in particular prostaglandins E₂.

The inflammatory phenomena are associated with many pathologies.

Representative thereof are, for example, sunburn, pruritus, erythema nodosum, urticaria, systemic mastocytosis, psoriasis, insect stings or other dermatological conditions such as atrophic polychondritis, erythermalgia or necrobiosis lipoidica. Also exemplary are disseminated lupus erythematosus, spondylarthropathies or articular attacks of chronic enteropathies.

Considerable research has to date been carried out in the pharmaceutical arts in quest of active agents for the treatment of inflammation.

In this respect, it has recently been proposed to administer a sufficient amount of a derivative of α-type melanocyte-stimulating hormone (α-MSH) or melanotropin and particularly the peptide containing the lysine-proline-valine tripeptide (U.S. Pat. No. 5,028,592 and U.S. Pat. No. 5,157,023).

However, it has been demonstrated that the optical form of the isomers comprising the composition of the tripeptide was of great importance. Thus, it has been shown that when the proline residue exists in the tripeptide in its dextrorotatory optical isomer form (Dpro), the tripeptide or the peptide containing the tripeptide lost all effectiveness in the treatment of inflammation (Hiltz et al., Peptides, Vol. 12, pp. 767-771 (1991)).

### SUMMARY OF THE INVENTION

It has now unexpectedly and surprisingly been discovered that a peptide containing the lysine-proline-valine tripeptide, in which the proline residue appears in the tripeptide in its dextrorotatory optical isomer form (DPro), or any functional biological equivalent thereof, is active for the treatment of inflammation.

By “functional biological equivalent” is intended a peptide which is functionally equivalent in terms of biological function, at least one of the amino acid residues of which may have been exchanged for an amino acid residue having a similar hydropathic index.

Briefly, thus, the present invention features pharmaceutical/cosmetic compositions for the treatment of inflammation, which comprise at least one peptide containing the lysine-proline-valine tripeptide, in which the proline residue exists in its dextrorotatory optical isomer form (DPro), or of any functional biological equivalent thereof.

### DETAILED DESCRIPTION OF BEST MODE AND SPECIFIC/PREFERRED EMBODIMENTS OF THE INVENTION

In the realm of amino acids, the geometry of the molecules is such that they can theoretically exist in the form of different optical isomers. Indeed, there exists a molecular configuration of the amino acid (aa) such that it rotates the plane of polarization of light to the right (dextrorotatory configuration or D-aa) and a molecular configuration of the amino acid (aa) such that it rotates the plane of polarization of light to the left (laevorotatory configuration or L-aa).

In nature, natural amino acids exist only in the laevorotatory configuration. Consequently, a peptide of natural origin will comprise only amino acids of L-aa type.

However, chemical synthesis in the laboratory permits preparation of amino acids having both possible configurations. From this base material, it is possible to incorporate, during the synthesis of peptides, amino acids both in the form of dextrorotatory or laevorotatory optical isomers.

It is thus possible to incorporate, during the synthesis of peptides, in addition to the D-proline (D-Pro) residue, lysine or valine amino acid residues which can equally well be in their D-lysine (D-Lys), L-lysine (L-Lys), D-valine (D-Val) or L-valine (L-Val) form.

Accordingly, the present invention features administration of a sufficient amount of the peptide as described above, for the treatment of inflammation, wherein the lysine or valine residues of the lysine-(D)proline-valine tripeptide constituting the peptide can equally well be in the form of dextrorotatory or laevorotatory optical isomers.

Exemplary peptides containing at least one of the following tripeptides include:

D-Lys-D-Pro-D-Val,

D-Lys-D-Pro-L-Val,

L-Lys-D-Pro-D-Val,

L-Lys-D-Pro-L-Val.

The tripeptide is advantageously situated at the C-terminal end of the peptide.

Preferably, the peptide according to the invention is the lysine-proline-valine tripeptide in which the proline residue exists in its dextrorotatory optical isomer form (DPro).

The peptide administered according to the invention also preferably contains the lysine-proline-valine tripeptide in which the lysine, proline and valine residues appear in the form of dextrorotatory optical isomers (DLys-DPro-DVal).

According to the invention, it is, of course, possible to use more than one peptide. In this event, the mixture of peptides may be composed of one of the possible combinations of the peptides described above.

Generally, as utilized herein, by the term “proline” is intended the proline residue in its dextrorotatory optical isomer form (DPro) and the term “peptide” comprehends both the “peptide containing the lysine-proline-valine tripeptide, or any functional biological equivalent” and the isolated “lysine-proline-valine tripeptide” in which the proline residue is in its dextrorotatory optical isomer form (DPro).

It may transpire that, for reasons of resistance to degradation, it is necessary to employ, according to the invention, a protected form of the peptide. The form of the protection must obviously be a biologically compatible form. Many biologically compatible protection forms may be envisaged, such as, for example, acylation or acetylation of the amino-terminal end or amidation of the carboxy-terminal end.

Thus, this invention also features administration of the subject peptide in a protected or unprotected form.

Preferably, a protective group is employed based either on acylation or acetylation of the amino-terminal end or on amidation of the carboxy-terminal end or, alternatively, on both.

The effective amount of active principle corresponds to the amount necessary to elicit the desired therapeutic effect.

More particularly, in the subject cosmetic compositions, the peptide is present in an amount such that the lysine-proline-valine tripeptide is at a concentration ranging from 10⁻¹²M to 10⁻³M and preferably from 10⁻⁹M to 10⁻⁴M.

Preferably, for the formulation of a medicament, the peptide is present in an amount such that the lysine-proline-valine tripeptide is employed at a concentration ranging from 10⁻¹²M to 1M and preferably from 10⁻⁶M and 10⁻¹M.

It would be apparent to one skilled in this art to adjust this amount of material depending on whether the peptide containing the lysine-proline-valine tripeptide is administered, or any functional biological equivalent thereof, or the lysine-proline-valine tripeptide, per se.

The compositions according to the invention can be administered parenterally, enterally or alternatively topically. The subject compositions are preferably administered topically.

The physiologically acceptable medium, i.e., carrier, diluent or vehicle, in which the peptide is formulated according to the invention can be anhydrous or aqueous. By “anhydrous medium” is intended a solvent medium containing less than 1% of water. This medium can be constituted of a solvent or of a mixture of solvents chosen more particularly selected from among C₂-C₄ lower alcohols, such as ethyl alcohol, alkylene glycols, such as propylene glycol, and the alkyl ethers of alkylene glycols or of dialkylene glycols, in which the alkyl or alkylene radicals contain from 1 to 4 carbon atoms. By “aqueous medium” is intended a medium constituted of water or a mixture of water and of another physiologically acceptable solvent selected, in particular, from among the organic solvents indicated above. In the latter instance, these other solvents, when they are present, constitute approximately 5% to 95% by weight of the composition.

It is possible for the physiologically acceptable medium to contain other adjuvants commonly used in the cosmetics or pharmaceutical arts, such as surface-active agents, thickening or gelling agents, cosmetic agents, preservatives or basifying or acidifying agents well known to the art, in amounts sufficient to provide the desired form of presentation, in particular of a more or less thickened lotion, of a gel, of an emulsion or of a cream. Forms pressurized in an aerosol or vaporized from a pump-action spray may also be used.

It is also possible to administer, in combination with the peptide, compounds which are already known to this art for their anti-inflammatory activity.

Exemplary thereof are the glucocorticoids, vitamin D and derivatives thereof and non-steroidal anti-inflammatories.

The peptides according to the invention can be administered by topical application of a cosmetic composition containing an effective amount of at least one peptide containing the lysine-proline-valine tripeptide in which the proline residue exists in its dextrorotatory optical isomer form (DPro) on a part of the body exhibiting symptoms of inflammation.

Thus, the present invention also features a cosmetic treatment, wherein a cosmetic composition containing an effective amount of at least one peptide containing the lysine-proline-valine tripeptide, in which the proline residue appears in its dextrorotatory optical isomer form (DPro), is topically applied onto the skin, onto the scalp and/or onto the mucous membranes exhibiting the symptoms of inflammation.

The cosmetic treatment according to the invention can be implemented, in particular, by applying the cosmetic compositions as described above via the usual techniques for administration of same. For example: application of anti-sun or sunscreen compositions or makeup removal milks, lotions, serums, gels or creams onto the skin or onto the scalp, of shampoos or, alternatively, of dentifrices onto the gums.

In order to further illustrate the present invention and the advantages thereof, the following specific examples are given, it being understood that same are intended only as illustrative and in nowise limitative.

In said examples to follow, all parts and percentages are given by weight with respect to the total weight of the particular composition.

### EXAMPLE 1

Dose-response activity of the Ac-KPV-NH₂* tripeptide with respect to the production of interleukin-1α in the supernatant of plucked hairs emanating from a patient afflicted with inflammatory alopecia.

Plucked hairs were removed from the region of the vertex of a volunteer suffering from inflammatory alopecia. They were placed in a Williams' survival medium E (marketed by Gibco BRL) containing penicillin G (100 units/ml), streptomycin S (100 μg/ml) and amphotericin (250 ng/ml), in the presence or in the absence (control) of the Ac-KPV-NH₂* tripeptide, synthesized to order by Neosystem S. A. (Strasbourg), at the doses indicated. After incubating for 20 hours, the culture supernatants were collected in a tube and then centrifuged for 5 minutes at 14,000 revolutions/minute (Eppendorff centrifuge, model 5415C). The supernatants were then collected in a clean tube and placed at 4° C.

The interleukin-1α concentration was then evaluated with respect to 100 μl of supernatant by means of a Biotrak ELISA kit marketed by Amersham, according to the manufacturer's instructions.

The results obtained were as follows:

TABLE I

| | Dose | IL-1α | % of inhibition |
|---|---|---|---|
| Control | | 21.8 pg/ml | |
| Ac—KPV—NH₂* | 10 μM | 6.1 pg/ml | 72% |
| Ac—KPV—NH₂* | 1 μM | 11.1 pg/ml | 49% |

### EXAMPLE 2

Inhibition of the expression of the messenger RNAs of proinflammatory and inflammatory cytokines in response to the Ac-KPV-NH₂* tripeptide.

Ten plucked hairs were removed from the region of the vertex of a volunteer suffering from inflammatory alopecia. These were placed in a Williams' survival medium E (marketed by Gibco BRL) containing penicillin G (100 units/ml), streptomycin S (100 μg/ml) and amphotericin (250 ng/ml), in the presence (treated batch) or in the absence (control) of the Ac-KPV-NH₂* tripeptide, synthesized to order by Neosystem S. A., (Strasbourg).

After incubating for 3 h, 30 min, the messenger RNAs corresponding to these two batches of hair were purified from a “quick prep mRNA purification kit” marketed by Pharmacia. DNAs complementary to these mRNAs were then prepared by means of a reverse transcription kit marketed by Pharmacia, the manufacturer's instructions being followed, and then subjected to a polymerization chain reaction (PCR) stage by using primers specific for the mRNAs of glyceraldehyde-3-phosphate dehydrogenase (GAPDH), of IL-1α, of the type-1 IL-1 receptor and of the type-2 IL-1 receptor. The amounts of amplified DNA were then evaluated by electrophoresis on 1.5% agarose gel in the presence of ethidium bromide. The intensity of the bands was estimated under ultraviolet radiation by means of a video camera and analytical software (Bioprofil®) which were marketed by Vilbert-Lourmat. The intensity of the bands obtained with the IL-1α, IL-1R1 and IL-1R2 primers was divided by the intensity of the bands obtained with the primers amplifying the internal standard GAPDH.

The results obtained are reported in the following Table II:

TABLE II

| % of | | | |
|---|---|---|---|
| expression | IL-1α | IL-1R1 | IL-1R2 |
| Control batch | 100% | 100% | 100% |
| Treated batch | 0% | 26% | 21% |

### EXAMPLE 3

Inhibiting effect of Ac-KPV-NH₂* on the expression of the mRNAs of IL-1α, calculated from the IL-1α/GAPDH ratio.

5 hairs were plucked from two different donors and were then incubated for 20 hours at 37° C. (5% CO₂) in Williams' medium E supplemented with antibiotics and with glutamine and in the presence of Ac-KPV-NH₂* at the concentrations indicated. A control was carried out in which there was no peptide added. The results obtained, expressed as % of the control, are reported in Table III below:

TABLE III

| Donors | | A | B |
|---|---|---|---|
| Control | | 100% | 100% |
| Ac—KPV—NH₂* | 10 μM | 28% | 28.7% |
| Ac—KPV—NH₂* | 1 μM | 51% | 62% |
| Ac—KPV—NH₂* | 0.1 μM | ND | 53% |

ND: Not determined

### EXAMPLE 4

Measurement of the inhibition of the production of PGE₂ by catagenic papilla cells cultured in vitro.

The cells (1,000 per well), at the 24th passage, were incubated in 199 medium marketed by Gibco in the presence of 1% foetal calf serum and of antibiotics. 20 hours later, the medium was replaced by an identical medium, but containing the tripeptides to be evaluated at a final concentration of 10 μM. 5 hours later, interleukin-1α was added at a final concentration of 10 ng/ml. 20 hours later, the PGE₂ levels produced by the cultured papilla cells were evaluated by means of a Biotrack kit marketed by Amersham, the manufacturer's instructions being followed. This method thus made it possible to evaluate the inhibiting effect of these tripeptides on the production of PGE₂ induced by a proinflammatory cytokine: interleukin-1α.

The results obtained are reported in the following Table IV:

TABLE IV

| | Dose | PGE₂ (pg/ml) | % inhibition |
|---|---|---|---|
| Control | | 9.2 | |
| IL-1α | | 100.2 | |
| Ac—KPV—NH₂* | 10 μM | 28.0 | 78% |
| Ac—K—P—V—NH₂** | 10 μM | 10.0 | 90% |
| Ac—K—(D)P—V—NH₂*** | 10 μM | 24.6 | 75% |

These results indicate, surprisingly, a comparable anti-inflammatory capacity for tripeptides containing either the (D)-Pro form or the natural (L)-Pro form.

### EXAMPLE 5

Examples of compositions containing the Ac-LPV-NH₂* tripeptides. These compositions were formulated by conventional preparative techniques and in particular by simple mixing of the ingredients.

Composition 1:
Spray:

| Ac—KPV—NH₂* | 5 × 10⁻⁶ | g |
|---|---|---|
| Minoxidil | 0.5 | g |
| 95° Ethanol | 55.1 | g |
| Propylene glycol | 22.8 | g |
| Fragrance | q.s. | |
| Demineralized water q.s. for | 100 | g |

Composition 2:
Daily Lotion:

| Ac—KPV—NH₂* | 12.5 × 10⁻⁶ | g |
|---|---|---|
| 2,4-Diaminopyrimidine 3-oxide | 0.75 | g |
| 95° Ethanol | 30 | g |
| Fragrance | q.s. | |
| Dyes | q.s. | |
| Demineralized water q.s. for | 100 | g |

Composition 3:
Liposomed gel:

| Natipide II¹ (i.e., 2 g of phospholipids) | 10 | g |
|---|---|---|
| Ac—KPV—NH₂* | 5 × 10⁻⁵ | g |
| Carbomer | 0.25 | g |
| Triethanolamine | q.s. pH = 7 | |
| Preservatives | q.s. | |
| Demineralized water q.s. for | 100 | g |

¹Water/alcohol/lecithin mixture marketed by Nattermann

Composition 4:
Niosomed gel:

| Chimexane NS¹ | 1.8 | g |
|---|---|---|
| Monosodium stearoylglutamate | 0.2 | g |
| Ac—KPV—NH₂* | 7.5 × 10⁻⁴ | g |
| Carbomer | 0.2 | g |
| Triethanolamine | q.s. pH = 7 | |
| Preservatives | q.s. | |
| Fragrances | q.s. | |
| Demineralized water q.s. for | 100 | g |

¹Nonionic surfactant marketed by Chimex.

Composition 5:
Niosomed lotion:

| Chimexane NL¹ | 0.475 | g |
|---|---|---|
| Cholesterol | 0.475 | g |
| Monosodium stearoylglutamate | 0.05 | g |
| Ac—KPV—NH₂* | 10⁻³ | g |
| Preservatives | q.s. | |
| Dyes | q.s. | |
| Fragrance | q.s. | |
| Demineralized water q.s. for | 100 | g |

¹Non-ionic surfactant marketed by Chimex.

Composition 6:
Care cream; O/W emulsion:

| Cetylstearyl alcohol/cetylstearyl | 5 | g |
|---|---|---|
| alcohol oxyethylenated with 33 mol | | |
| of ethylene oxide (80/20) | | |
| Glyceryl monostearate | 1.5 | g |
| Cetyl alcohol | 0.75 | g |
| Liquid petrolatum | 10 | g |
| Polydimethylsiloxane | 0.75 | g |
| Glycerol | 4 | g |
| Preservatives | q.s. | |
| Ac—KPV—NH₂* | 5 × 10⁻³ | g |
| Demineralized water q.s. for | 100 | g |

Composition 7:
Solution injectable via intradermal route:

| | |
|---|---|
| Ac—KPV—NH₂* | 0.7 mg |
| Physiological serum | 1 ml |
| (NaCl 9 g/H₂O q.s. for 100 ml) q.s. for | |

*Acetyl-(D)Lys-(D)Pro-(D)Val-NH₂
** Acetyl-(L)Lys-(L)Pro-(L)Val-NH₂
*** Acetyl-(L)Lys-(D)Pro-(L)Val-NH₂

While the invention has been described in terms of various preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A method for the effective treatment of an inflammation state afflicting a mammalian organism, comprising administering to said organism a therapeutically effective amount of a lysine-proline-valine tripeptide, the proline moiety of which exists in its dextrorotatory optical isomer form (DPro), formulated into a physiologically/pharmaceutically acceptable medium therefor.

2. The method as defined in claim 1, wherein said lysine-proline-valine tripeptide includes at least one protective group therefor.

3. The method as defined in claim 2, wherein said at least one protective group comprises an acyl, acetyl and/or amido group.

4. The method as defined in claim 1, wherein said lysine-proline-valine tripeptide is at a concentration ranging from 10⁻¹²M to 10⁻³M.

5. The method as defined in claim 4, wherein said lysine-proline-valine tripeptide is at a concentration ranging from 10⁻⁹M to 10⁻⁴M.

6. The method as defined in claim 1, wherein said lysine-proline-valine tripeptide is at a concentration ranging from 10⁻¹²M to 1M.

7. The method as defined in claim 6, wherein said lysine-proline-valine tripeptide is at a concentration ranging from 10⁻⁶M to 10⁻¹M.

8. The method as defined in claim 1, wherein said lysine-proline-valine tripeptide is in the form of a lotion, gel, milk, serum, cream, sunscreen, emulsion, shampoo, dentifrice, ointment, aerosol or spray.

9. The method as defined in claim 1, comprising parenterally, enterally or topically administering said lysine-proline-valine tripeptide.

10. The method as defined in claim 9, comprising topically applying said lysine-proline-valine tripeptide to the skin, scalp and/or mucous membranes of said mammalian organism.

11. The method as defined in claim 1, further comprising administering to said organism an effective anti-inflammatory amount of at least one glucocorticoid, vitamin D or derivative thereof, and/or non-steroidal anti-inflammatory agent.

12. The method as defined in claim 1, wherein in said lysine-proline-valine tripeptide is selected from the group consisting of D-Lys-D-Pro-D-Val, D-Lys-D-Pro-L-Val, L-Lys-D-Pro-D-Val and L-Lys-D-Pro-L-Val.

13. A method for inhibiting the expression of the messenger RNAs of proinflammatory and inflammatory cytokines in a mammalian organism in need of said inhibition, comprising administering to said organism a therapeutically effective amount of a lysine-proline-valine tripeptide, the proline moiety of which exists in its dextrorotatory optical isomer form (DPro), formulated into a physiologically/pharmaceutically acceptable medium therefor.

14. The method as defined in claim 13, wherein in said lysine-proline-valine tripeptide is selected from the group consisting of D-Lys-D-Pro-D-Val, D-Lys-D-Pro-L-Val, L-Lys-D-Pro-D-Val and L-Lys-D-Pro-L-Val.

15. The method as defined in claim 13, wherein said lysine-proline-valine tripeptide includes at least one protective group therefor.

16. The method as defined in claim 15, wherein said at least one protective group comprises an acyl, acetyl and/or amido group.

17. The method as defined in claim 13, wherein said lysine-proline-valine tripeptide is at a concentration ranging from 10⁻¹²M to 10⁻³M.

18. The method as defined in claim 17, wherein said lysine-proline-valine tripeptide is at a concentration ranging from 10⁻⁹M to 10⁻⁴M.

19. The method as defined in claim 13, wherein said lysine-proline-valine tripeptide is at a concentration ranging from 10⁻¹²M to 1M.

20. The method as defined in claim 19, wherein said lysine-proline-valine tripeptide is at a concentration ranging from 10⁻⁶M to 10⁻¹M.

21. The method as defined in claim 13, comprising parenterally, enterally or topically administering said lysine-proline-valine tripeptide.

22. The method as defined in claim 13, further comprising administering to said organism an effective anti-inflammatory amount of at least one glucocorticoid, vitamin D or derivative thereof, and/or non-steroidal anti-inflammatory agent.
